(19) United States
(12) Patent Application Publication
(10) Pub. No.: US 2017/0239023 A1
(43) Pub. Date: Aug. 24, 2017

(54) SYSTEM AND METHOD OF FORMING DENTURES

(71) Applicant: Justin Spencer Marks, Astoria, NY (US)

(72) Inventor: Justin Spencer Marks, Astoria, NY (US)

(21) Appl. No.: 15/428,533

(22) Filed: Feb. 9, 2017

Related U.S. Application Data
(60) Provisional application No. 62/299,198, filed on Feb. 24, 2016.

(51) Int. Cl. A61C 13/00; A61C 9/00; A61C 13/36; A61C 13/01; A61C 13/08; A61B 1/24; A61C 13/103

(52) U.S. Cl. CPC A61C 13/0004; A61B 1/24; A61C 9/0053; A61C 13/0019; A61C 13/1009; A61C 13/01; A61C 13/08; A61C 13/1016

(57) ABSTRACT

A method of forming a denture is provided. The method includes the steps of: creating a digital data recording of an oral cavity of a patient; merging the digital data recording with additional digital data depicting a gingiva portion of either an upper or a lower denture via a software running on a computer. The gingiva portion includes plurality of tooth sockets and at least one of a male and a female connector disposed therein. The method further includes the step of constructing a first denture portion by a 3D printer. The first denture portion resembles the gingiva portion of the merged digital data.

### CROSS-REFERENCE TO RELATED APPLICATION

This application claims the benefit of priority of U.S. provisional application No. 62/299,198, filed Feb. 24, 2016, the contents of which are herein incorporated by reference.

### BACKGROUND OF THE INVENTION

The present invention relates to a system and method of forming dentures.

Previous methods for fabricating removable dentures involve time consuming analog techniques. Digital design software exists for the creation of dentures but previous manufacturing methods do not allow for efficient automated techniques to build a physical object from the digital design. Furthermore, denture bases that are created through additive manufacturing often do not form a chemical bond with prosthetic denture teeth that are fabricated through other means.

As can be seen, there is a need for an improved system and method of forming a denture base that mechanically fastens to denture teeth.

### SUMMARY OF THE INVENTION

In one aspect of the present invention, a method of forming a denture comprises: creating a digital data recording of an oral cavity of a patient; merging the digital data recording with additional digital data depicting a gingiva portion of at least one of an upper and a lower denture via a software running on a computer, wherein the gingiva portion comprises a plurality of tooth sockets and at least one of a male and a female connector disposed with the plurality of tooth sockets; and constructing a first denture portion by a 3D printer wherein the first denture portion resembles the gingiva portion of the merged digital data.

In another aspect of the present invention, a method of forming a denture comprises: creating a digital data recording of an oral cavity of a patient; merging the digital data recording with additional digital data depicting a gingiva portion of at least one of an upper and a lower denture via a software running on a computer, wherein the gingiva portion comprises a plurality of tooth sockets each comprising at least one of a male and female connector disposed therein; constructing a first denture portion by a 3D printer wherein the first denture portion resembles the gingiva portion of the merged digital data; providing a second denture portion, wherein the second denture portion resembles a plurality of teeth, wherein each of the plurality of teeth comprise the other of the male and the female connector; and mechanically fastening the first denture portion to the second denture portion by connecting the male connectors to the female connectors.

These and other features, aspects and advantages of the present invention will become better understood with reference to the following drawings, description and claims.

### BRIEF DESCRIPTION OF THE DRAWINGS

FIG. 1 is a block diagram of an embodiment of the present invention;

FIG. 2 is a front elevation view of an embodiment of the scanning step;

FIG. 3 is a perspective view of a 3D printer creating a denture base;

FIG. 4 is an exploded front view of an embodiment of the present invention;

FIG. 5 is section view of an embodiment of the present invention; and

FIG. 6 is a front elevation view of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a removable or fixed denture prosthesis fabricated from thermoplastic materials using a fused deposition modeling additive manufacturing process. The present invention allows individuals to 3D print a denture base from a digital design using thermoplastic materials. The denture base may then be combined with prefabricated or custom manufactured prosthetic teeth to complete the dental restoration.

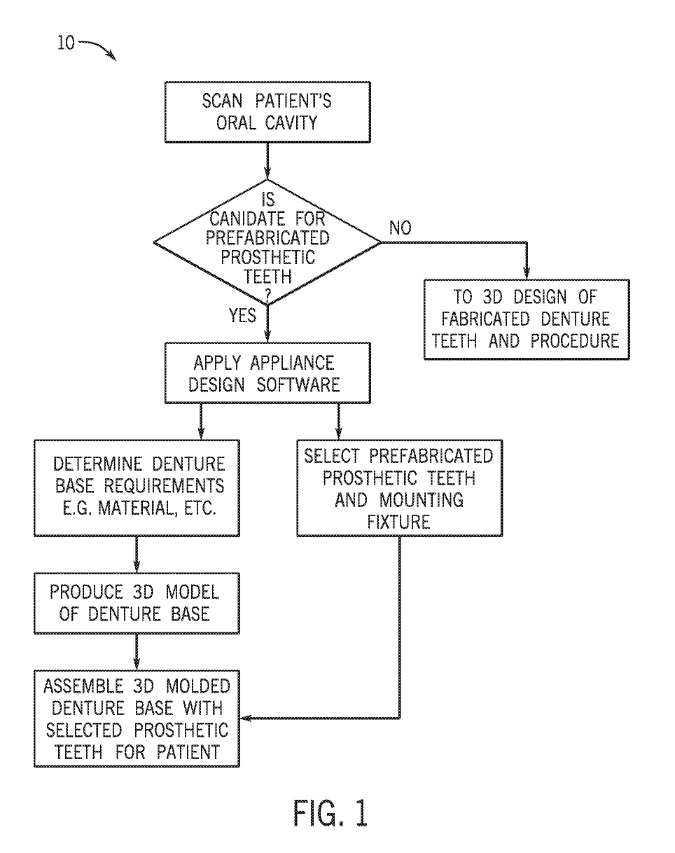

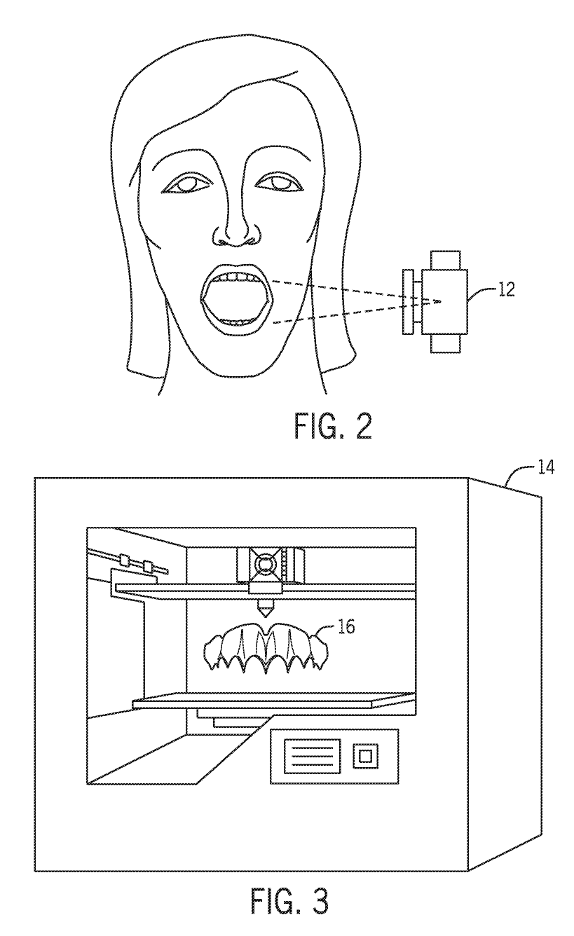

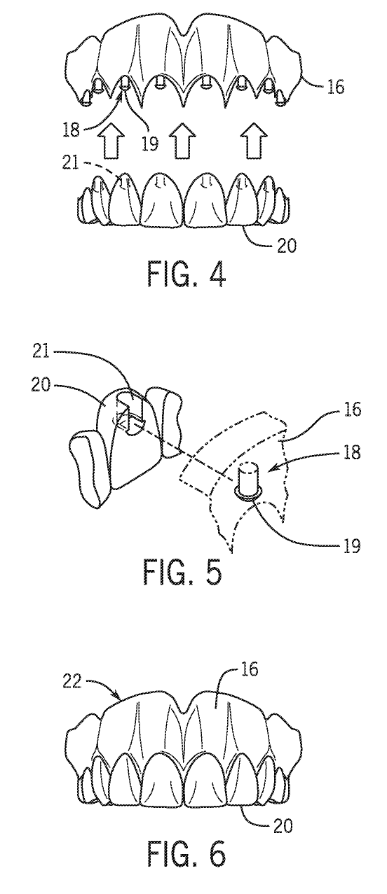

Referring to FIGS. 1 through 6, the present invention includes a method of forming a denture 22. The method includes the steps of: creating a digital data recording of an oral cavity of a patient; merging the digital data recording with additional digital data depicting a gingiva portion of either an upper or a lower denture via a software running on a computer. The gingiva portion includes plurality of tooth sockets and at least one of a male and a female connector disposed with the plurality of tooth sockets. The method further includes the step of constructing a first denture portion 16 by a 3D printer 14. The first denture portion 16 resembles the gingiva portion of the merged digital data.

The digital data recording may be made of the oral cavity without teeth, or with partial teeth-deficiency. The digital data recording may be created by using a scanner 12 to directly scan the oral cavity. Alternatively, an impression of the oral cavity may be made or a gypsum cast from an impression of the oral cavity. Moreover, a digital data recording is also possible with 3D CT, respectively a 3D SCAN method. Following the digital recording, additional information is acquired regarding the position (articulation) of the mandible (mandibula), respectively the upper jaw (maxilla) of the patient.

The information is processed using software run on a computer and the virtual digital image of the removable dentures (prostheses) is modelled. The digital data recording is forwarded to the computer. The additional digital data depicting a gingiva portion of either an upper or a lower denture are merged so that additional parameters are added to the digital data recording. The computer thereby creates the digital design of the first denture portion 16. The software slices the digital design into layers and the data is transmitted to the 3D printer 14. The 3D printer 14 then uses the digital design to create the first denture portion 16. A physical model of the denture is extruded from the printer, layer by layer, until the first denture portion 16 is complete.

The present invention may further include a second denture portion 20 resembling a plurality of teeth. The second denture portion 20 includes the other of the male and female connectors. In certain embodiments, the second denture portion 20 may be prefabricated or may be constructed using the 3D printer 14. The second denture portion 20 is mechanically fastened to the first denture portion 16 by connecting the male connector to the female connector and joining them with a resin or cement. A placement jig may be used to facilitate proper alignment of the artificial teeth to the gingiva portion.

The first and second denture portions 16, 20 may be formed of a thermoplastic. In such embodiments, the 3D printer 14 of the present invention may utilize fused deposition modeling. In this technique, filaments of heated thermoplastic are extruded from a tip that moves in the x-y plane. The controlled extrusion head deposits very thin beads of material onto the build platform to form the first layer. The platform is maintained at a lower temperature, so that the thermoplastic quickly hardens. After the platform lowers, the extrusion head deposits a second layer upon the first. Supports are built along the way, fastened to the part either with a second, weaker material or with a perforated junction. The present invention may use other 3D printing techniques, such as, but not limited to, selective laser sintering, solid ground curing, ink jet printing and the like.

In certain embodiments, the first denture portion 14 includes the male connector and the second denture portion 20 includes the female connector. The male connector may include a plurality of pegs 18 each disposed within one of the plurality of tooth sockets. Each of the plurality of pegs 18 may include a shaft and a head. The head may include a larger diameter than the shaft. The female connector may include a plurality of slots each sized to receive and mechanically retain a peg within. For example, the head may snap into the slot and the slot may prevent the head from passing back through the slot. Therefore, the peg 18 mechanically fastens within the slot, thereby mechanically fastening the first denture portion 14 to the second denture portion 20.

It should be understood, of course, that the foregoing relates to exemplary embodiments of the invention and that modifications may be made without departing from the spirit and scope of the invention as set forth in the following claims.

What is claimed is:

1. A method of forming a denture comprising:
creating a digital data recording of an oral cavity of a patient;
merging the digital data recording with additional digital data depicting a gingiva portion of at least one of an upper and a lower denture via a software running on a computer, wherein the gingiva portion comprises a plurality of tooth sockets and at least one of a male and a female connector disposed with the plurality of tooth sockets; and
constructing a first denture portion by a 3D printer wherein the first denture portion resembles the gingiva portion of the merged digital data.

2. The method of claim 1, wherein the step of creating the digital data recording of the oral cavity comprises the step of: scanning the oral cavity of the patient.

3. The method of claim 1, further comprising the steps of:
providing a second denture portion, wherein the second denture portion resembles a plurality of teeth, wherein the second denture portion comprise the other of the male and the female connector; and
mechanically fastening the first denture portion to the second denture portion by connecting the male connector to the female connector.

4. The method of claim 3, wherein the step of providing a second denture portion comprises constructing the second denture portion by a 3D printer.

5. The method of claim 3, wherein the first denture portion comprises the male connector and the second denture portion comprises the female connector.

6. The method of claim 5, wherein the male connector comprises a plurality of pegs each disposed within one of the plurality of tooth sockets and the female connector comprises a plurality of slots each sized to receive and mechanically retain a peg of the plurality of pegs therein.

7. The method of claim 3, further comprising the step of:
adding at least one of a resin and a cement to further bond the first denture portion and the second denture potion together.

8. The method of claim 1, wherein the first denture portion is formed by a thermoplastic and the step of constructing a first denture portion by a 3D printer is performed by fused deposition modeling.

9. A method of forming a denture comprising:
creating a digital data recording of an oral cavity of a patient;
merging the digital data recording with additional digital data depicting a gingiva portion of at least one of an upper and a lower denture via a software running on a computer, wherein the gingiva portion comprises a plurality of tooth sockets each comprising at least one of a male and female connector disposed therein;
constructing a first denture portion by a 3D printer wherein the first denture portion resembles the gingiva portion of the merged digital data;
providing a second denture portion, wherein the second denture portion resembles a plurality of teeth, wherein each of the plurality of teeth comprise the other of the male and the female connector; and
mechanically fastening the first denture portion to the second denture portion by connecting the male connectors to the female connectors.

10. The method of claim 9, wherein the step of providing a second denture portion comprises constructing the second denture portion by a 3D printer.

11. The method of claim 9, wherein the first denture portion comprises the male connectors and the second denture portion comprises the female connectors.

12. The method of claim 11, wherein the male connectors are pegs and the female connectors are slots sized to receive and mechanically retain the pegs therein.

13. The method of claim 9, wherein the first denture portion is formed by a thermoplastic, and the step of constructing a first denture portion by a 3D printer is performed by fused deposition modeling.

14. The method of claim 9, further comprising the step of:
adding at least one of a resin and a cement to further bond the first denture portion and the second denture potion together.
